(19) United States
(12) Patent Application Publication
(10) Pub. No.: US 2012/0191170 A1
(43) Pub. Date: Jul. 26, 2012

(54) BIFURCATED FLOW DEVICE FOR CARDIO-PULMONARY ASSIST OR SUPPORT AND ASSOCIATED METHODS

(76) Inventor: Gordon Cohen, Mercer Island, WA (US)

(21) Appl. No.: 13/358,465

(22) Filed: Jan. 25, 2012

Related U.S. Application Data
(62) Division of application No. 11/832,433, filed on Aug. 1, 2007, now abandoned.
(60) Provisional application No. 60/835,227, filed on Aug. 2, 2006.

(51) Int. Cl. A61F 2/06

(52) U.S. Cl. 623/1.3; 623/1.35

(57) ABSTRACT

A graft useable with a blood flow device that allows the use of a single blood flow device to simultaneously provide blood flow to the systemic and pulmonary circulations. The graft has a tubular inlet portion coupleable to the outlet of the blood-flow assist device and configured to receive blood flow therethrough. A tubular systemic portion is connected to the inlet portion and configured to be connected to the systemic circulation of the patient to direct the first portion of the blood flow into the systemic circulation at a first flow rate. The graft has a tubular pulmonary portion connected to the inlet portion and configured to be connected to the pulmonary circulation of the patient to direct a second portion of the blood flow into the pulmonary circulation substantially simultaneous with delivery of the first portion of the blood flow into the systemic circulation.

### CROSS-REFERENCE TO RELATED APPLICATION

The present application is a divisional patent application of U.S. patent application Ser. No. 11/832,433, titled BIFURCATED FLOW DEVICE FOR CARDIO-PULMONARY ASSIST OR SUPPORT AND ASSOCIATED METHODS, filed Aug. 1, 2007, which non-provisional patent application that hereby claims priority to U.S. Provisional Patent Application No. 60/835,227, filed Aug. 2, 2006, and both of which is are incorporated herein by reference in their its entirety, and to which priority is hereby claimed.

### TECHNICAL FIELD

Embodiments of the present invention relate to devices and methods for assisting blood flow relative to the heart.

### BACKGROUND

End-stage heart failure is a lethal disease. Treatment for this has traditionally been limited to heart transplantation. In the past decade there has been an emergence of mechanical devices that can be used to help support the systemic (body) circulation when the heart fails. Examples of such devices are described in U.S. patent application Ser. No. 10/171,023, entitled Implantable Heart Assist System and Method of Applying Same, filed Jun. 11, 2002, and U.S. patent application Ser. No. 11/134,226, entitled Replaceable Expandable Transmyocardial Ventricular Assist Device, filed May 20, 2005, both of which are incorporated herein in their entireties by reference thereto. Mechanical assist devices have evolved and have reached the point where they can now be reduced in size and become fully implantable. Despite the reduction in size the devices can only be implanted to support the systemic circulation. The devices remain too large in size so that two devices can be implanted to support both the pulmonary (lung) and the systemic circulation. Hence, the need for a total artificial heart has been identified and developed but has not been successfully employed with satisfactory long term results.

### BRIEF DESCRIPTION OF THE DRAWINGS

FIG. 1 is a schematic illustration of a human's circulatory system, a blood-flow assist device and bifurcated tubular graft in accordance with at least one embodiment of the present invention.

FIG. 2 is an enlarged schematic view of the bifurcated graft of FIG. 1.

FIG. 3 is an enlarged schematic view of the bifurcated graft in accordance with another embodiment of the present invention.

### DETAILED DESCRIPTION

Aspects of the present invention are directed generally toward a graft for use with a single blood flow device that allows the use of a single blood flow device to simultaneously provide blood flow to the systemic and pulmonary circulations of a patient. One aspect of the invention is directed toward a bifurcated graft for use with a blood-flow assist device. The blood-flow assist device is coupleable to the heart of a patient to receive blood, and the device has a blood flow outlet. The graft comprises an inlet portion configured to be coupled to the outlet of the blood-flow assist device and configured to receive blood flow therethrough. The graft has a systemic portion coupled to the grafts inlet portion and configured to receive a first portion of the blood flow from the inlet portion. In one embodiment, the systemic portion has a systemic outlet configured to be connected to a first artery in the patient's systemic circulation to direct the first portion of the blood flow into the artery. The systemic portion provides a first resistance to blood flow therethrough so the blood flows from the systemic portion at a blood flow rate and at a first pressure suitable with the systemic circulation. The graft also has a pulmonary portion coupled to the inlet portion and configured to receive a second portion of the blood flow from the inlet portion. The pulmonary portion has a pulmonary outlet configured to be connected to another artery in the patient's pulmonary circulation to direct the second portion of the blood flow into the artery substantially simultaneous with delivery of the first portion of the blood flow to the first artery The pulmonary portion provides a second resistance to the blood flow, wherein the second resistance is different than the first resistance. Accordingly, the blood flows from the pulmonary portion at a second pressure suitable with the pulmonary circulation

Other aspects of the invention are directed toward a graft for use with a blood-flow assist device coupleable to the heart of a patient and having a blood flow outlet. The graft comprises a tubular inlet portion coupleable to the outlet of the blood-flow assist device and configured to receive a blood flow therethrough. The graft has a tubular systemic portion connected the inlet portion and configured to be connected to the systemic circulation of the patient. The systemic portion has a first resistance to blood flow so as to direct the first portion of the blood flow into the systemic circulation at a flow rate and at a first pressure suitable with the systemic circulation. The graft has a tubular pulmonary portion connected to the inlet portion and configured to be connected to the pulmonary circulation of the patient. The pulmonary portion has a second resistance to blood flow, so as to direct a second portion of the blood flow into the pulmonary circulation substantially simultaneous with delivery of the first portion of the blood flow into the systemic circulation. The blood flows from the pulmonary portion at a second pressure suitable with the pulmonary circulation. In one embodiment, the first pressure is different than the second pressure.

Still other aspects of the invention are directed toward a cardio-pulmonary assistance system for use with a heart, a systemic circulation, and a pulmonary circulation of a patient. The system comprises a blood-flow assist device having a first inlet portion and an outlet portion. The inlet portion is coupled to the heart and configured to receive a flow of blood therein. The system includes a tubular graft shaped and sized to be surgically implanted in the patient. The graft comprises a tubular second inlet portion coupled to the outlet of the blood-flow assist device and configured to receive the blood flow therethrough. The graft has a systemic branch coupled to the second inlet portion configured to be connected to the systemic circulation of the patient to direct a first portion of the blood flow into the systemic circulation at a flow rate and a first pressure suitable with the systemic circulation. The graft also has a pulmonary branch coupled to the inlet portion and configured to be connected to the pulmonary circulation of the patient to direct a second portion of the blood flow into the pulmonary circulation substantially simultaneous with delivery of the first portion of the blood flow to the systemic circulation. The pulmonary branch is configured to deliver the blood flow into the pulmonary circulation at a flow rate and at a second pressure suitable with the pulmonary system.

Other aspects of the invention are directed toward a method of providing cardio-pulmonary assistance (including partial or full support) for a patient having a heart, a systemic circulation, and a pulmonary circulation. The method of one embodiment comprises providing a blood-flow assist device having an inlet portion and an outlet portion. The inlet portion is coupleable to the heart and configured to receive a flow of blood therefrom. The method comprises providing a tubular graft configured to carry the blood flow therethrough. The graft is shaped and sized to be surgically implanted in the patient. The graft has an inlet portion coupled to the outlet of the blood-flow assist device and configured to receive the blood flow therethrough. The graft has a systemic branch coupled to the graft's inlet portion and configured to be connected to the systemic circulation of the patient to direct a first portion of the blood flow into the systemic circulation at a flow rate and at a first pressure suitable with the systemic circulation. The graft has a pulmonary branch coupled to the graft's inlet portion and configured to be connected to the pulmonary circulation of the patient to direct a second portion of the blood flow into the pulmonary circulation substantially simultaneous with delivery of the first portion of the blood flow to the systemic circulation. The pulmonary branch is configured to deliver the second portion of the blood flow into the pulmonary circulation at a determined flow rate and at a second pressure suitable with the pulmonary circulation. In one embodiment, the flow rates through the pulmonary and systemic branches are substantially equal. The systemic and pulmonary branches can be configured so the flow resistance in each branch is different from each other. Accordingly, the branches can be configured so the blood flow from the systemic branch is provided to the systemic circulation at a first pressure and the blood flow from the pulmonary branch is provided to the pulmonary circulation at a second pressure different than the first pressure.

Other aspects of the invention are directed to a method of providing cardio-pulmonary assistance (including partial or full support) for a patient having a heart, a systemic circulation, and a pulmonary circulation. One embodiment of the invention comprises surgically installing in a patient a blood-flow assist device having an inlet portion and an outlet portion, wherein the inlet portion is coupled to the heart and is positioned to direct a flow of blood out the outlet portion. The method includes providing a tubular graft configured to carry the blood flow therethrough from the blood-flow assist device and delivering the blood flow into the systemic and pulmonary circulations. The graft has a graft inlet portion, a systemic branch coupled to the graft inlet portion, and a pulmonary branch coupled to the graft inlet portion. The method includes coupling the graft inlet portion to the outlet portion of the blood-flow assist device to allow the blood flow from the blood-flow assist device to flow into the graft. The method also includes attaching the systemic branch of the graft to the systemic circulation to allow a first portion of the blood flow to pass from the systemic branch into the systemic circulation. The method includes attaching the pulmonary branch of the graft to the pulmonary circulation to allow a second portion of the blood flow to pass from the pulmonary branch into the pulmonary circulation substantially simultaneous with the first portion of the blood flow moving into the systemic circulation.

Various embodiments of the invention will now be described. The following description provides specific details for a thorough understanding and enabling description of these embodiments. One skilled in the art will understand, however, that the invention may be practiced without many of these details. Additionally, some well-known structures or functions may not be shown or described in detail, so as to avoid unnecessarily obscuring the relevant description of the various embodiments.

The terminology used in the description presented below is intended to be interpreted in its broadest reasonable manner, even though it is being used in conjunction with a detailed description of certain specific embodiments of the invention. Certain terms may even be emphasized below; however, any terminology intended to be interpreted in any restricted manner will be overtly and specifically defined as such in this Detailed Description section. For example, as used herein ventricle assist includes partial and full support for ventricular action to facilitate the flow of blood to a patient's systemic and/or pulmonary circulatory systems. In addition, embodiments will be discussed below with reference to blood and blood flow, which will include natural blood or other oxygen or gas-carrying natural blood-substitutes.

References throughout the specification to “one embodiment” or “an embodiment” means that a particular feature, structure, or characteristic described in connection with the embodiment and included in at least one embodiment of the present invention. Thus, the appearances of the phrase “in one embodiment” or “in an embodiment” in various places throughout the specification are not necessarily all referring to the same embodiment. Furthermore, the particular features, structures, or characteristics may be combined in any suitable manner in one or more embodiments.

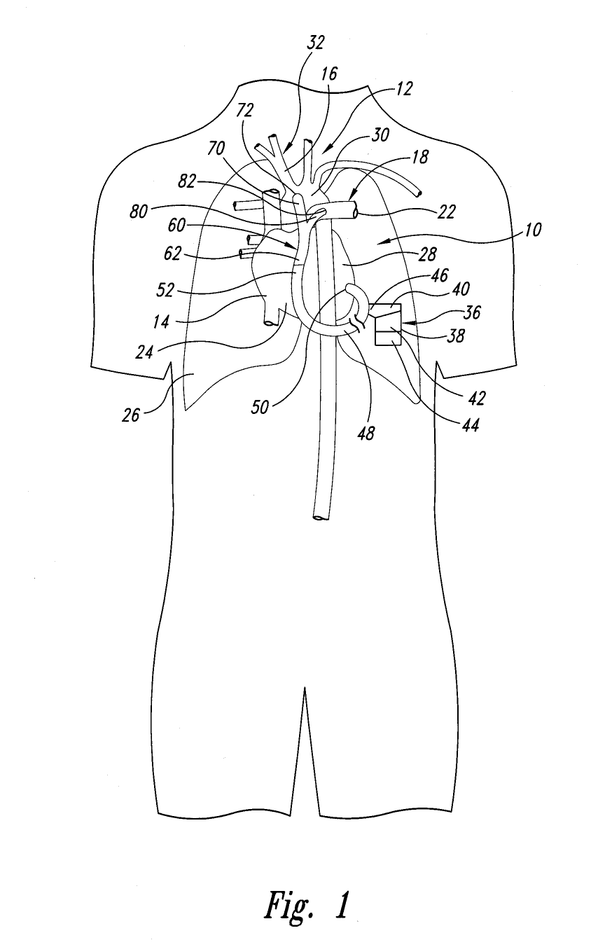

FIGS. 1-3 illustrate various features of a cardio-pulmonary assistance system 10 with a bifurcated graft 20 in accordance with embodiments of the invention. FIG. 1 is a schematic illustration of a portion of a human's circulatory system 12. The circulatory system includes a heart 14 connected to a pulmonary circulation 16 and a systemic circulation 18. The pulmonary circulation 16 carries blood from the heart 14 to the person's lungs 26 (where the blood is oxygenated and then returned to the heart). More specifically, the pulmonary circulation 16 includes the pulmonary artery 22 connected to the right ventricle 24 of the heart and coupled to the person's lungs 26. Accordingly, a healthy heart 14 would normally pump a flow of blood from the heart's right ventricle 24 into the pulmonary artery 22, which carries the blood flow toward the lungs.

The systemic circulation 18 carries oxygenated blood from the heart 14 to the rest of the body. The systemic circulation 18 is connected to the left ventricle 28 of the heart 14, which receives the returning flow of oxygenated blood. A healthy heart would pump a flow of oxygenated blood from the left ventricle 28 into the aorta 30 of the systemic system 18. The aorta 30 is connected to a plurality of major aorta branches 32, which are coupled to other arteries in the systemic circulation 18 that carry the oxygenated blood flow to the other parts of the person's body.

The cardio-pulmonary assistance system 10 of the illustrated embodiment includes a blood-flow assist device 36 connected to the heart 14 (i.e., an acute or chronic failing heart). The blood-flow assist device 36 is configured to provide assistance to the heart 12 and to pump the flow of blood through the circulatory system 12. The blood-flow assist device 36 can provide full or partial support to the heart 14 to decrease the load on the heart to achieve suitable blood flow through the circulatory system 12 for sustained life. The blood-flow assist device 36 can be a surgically implanted pump device that delivers blood flow, including augmenting blood flow or enhancing circulation, to assist or replace at least a portion of the pumping action of the failing heart. As an example, the blood-flow assist device 36 in the illustrated embodiment is a Ventricle Assist Device (VAD) 38. In one embodiment, the VAD 38 can be a commercially available device, such as a Debakey Micromed device or other axial flow device. Other examples of VADs include a Novacor unit manufactured by World Heart, a Heartmate unit manufactured by Thoratec, although other blood-flow assist devices could be used in other embodiments. In other examples, the blood-flow assist device 36 can be an artificial heart.

In the illustrated embodiment, the VAD 38 has a pump system 40, a control system 42 and a power supply 44. The pump system 40 has a pump 46 operatively connected to a tubular member 48 shaped and sized to carry blood flow through the VAD. The pump 46 can be, as an example, an axial flow device, a pulsification device, a non-pulsification device, a synchronous device, or a non-synchronous device. The pump 46 can be an axial pump, a sac diaphragm, a pusher-plate, centrifugal-style pump device, or other suitable pump device to drive or otherwise assist the blood flow. The pump system 40 can be adjusted locally or remotely via the control system 42 to provide a selected blood flow rate through the tubular member 48.

The pump 46 is operatively connected to the tubular member 48, which has an inlet portion 50 coupleable to the heart. The tubular member 48 has an outlet portion 52 through which the blood flow exits the VAD 38. In the illustrated embodiment, the VAD 38 is a Left Ventricle Assist Device (LVAD) wherein the inlet portion 50 of the tubular member 48 is connected to the heart's left ventricle. While the illustrated embodiment uses a LVAD, the VAD 38 in other embodiments can be a Right Ventricle Assist Device (RVAD) wherein the inlet portion 50 of the tubular member 48 is connected to the hearts right ventricle 24. Accordingly, blood flow is pumped or drawn from the heart 14 into the inlet portion 50, through the tubular member 48 and out of the VAD 38 through the outlet portion 52.

The cardio-pulmonary assist device 10 of the illustrated embodiment includes a bifurcated tubular graft 60 coupled to the blood-flow assist device 36 to receive the blood flow from the tubular member 48. The bifurcated graft 60 in accordance with one embodiment has one inlet and two outlets and is configured to simultaneously deliver blood flow to assist or support the systemic and pulmonary circulations while using a single blood flow assist device.

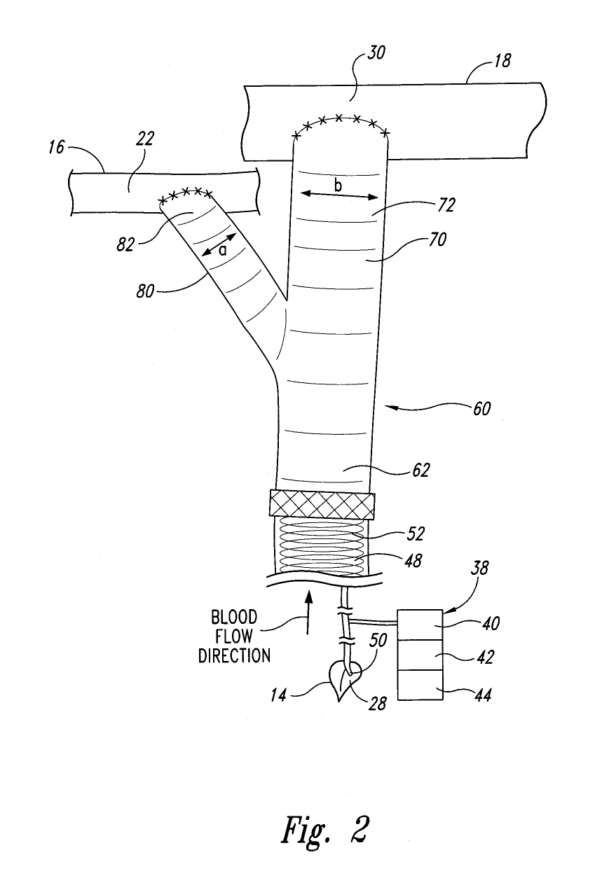

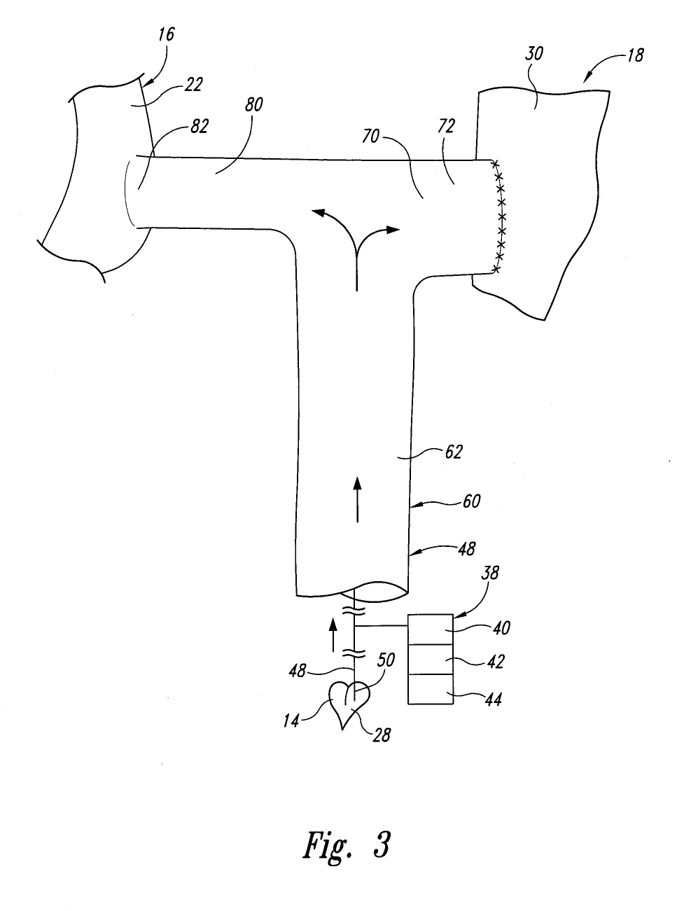

FIG. 2 is an enlarged schematic view of the bifurcated graft 60 of FIG. 1. FIG. 3 is an enlarged schematic view of the graft 60 in accordance with another embodiment. The graft 60 includes an inlet portion 62 coupled to the blood-flow assist device 36 and configured to receive the blood flow. The inlet portion 62 is a tubular structure connected to the outlet portion 52 of the tubular member 48. In one embodiment, the inlet portion 62 of the graft is connected directly to the tubular member 48, such as by a mechanical interlocking device or other device, to form a fluid-tight and air-tight seal therebetween to allow the blood flow to smoothly pass into the graft at a selected flow rate and pressure. In one embodiment, the inlet portion 62 can be removably connected to the VAD 38 in the event that the VAD or a portion of the VAD requires repair, maintenance, or replacement. In another embodiment, the inlet portion 62 of the graft 60 can be integrally connected to the outlet portion 52 of the tubular device 48 of the VAD 38. In yet another embodiment, the inlet portion 62 of the graft 60 can be connected to the tubular member 48 of the VAD 38 via an intermediate tubular structure or other intermediary device, such as an oxygenator, a pressure measurement device, flow measurement device, a coupling device, or other device or intermediate structure.

The graft 60 has a tubular systemic branch 70 connected to the inlet portion 62 and configured to receive a first portion of the blood flow from the graft's inlet portion. The systemic branch 70 of the illustrated embodiment has a free end 72 connectable to a portion of the patient's systemic circulation 18 so that the first portion of the blood flow is directed from the systemic branch into the systemic circulation. The systemic branch 70 is shaped and sized to provide a flow resistance so the first portion of the blood flow is provided to the systemic circulation at a flow rate and with a pressure suitable for the patient's systemic circulation. In the illustrated embodiment, the free end 72 of the systemic branch 70 is sutured or otherwise surgically attached to a portion of the patient's aorta 30, such as to the ascending aorta, to provide a fluid and air tight interconnection therebetween. In other embodiments, the systemic branch 70 can be connected to another portion of the systemic circulation 18, such as one of the major aorta branches or other selected artery in the systemic circulation. In another embodiment the systemic branch 70 can be connected to another conduit attached to the systemic circulation.

The graft 60 also has a pulmonary branch 80 connected to the graft's inlet portion 62 and configured to receive a second portion of the blood flow from the inlet portion. The pulmonary branch 80 of the illustrated embodiment has a free end 82 connected to a portion of the patient's pulmonary circulation 16 so that the second portion of the blood flow is directed into the pulmonary circulation. The pulmonary branch 80 is shaped and sized to provide a resistance to the blood flow that is different than the flow resistance of the systemic branch 70. Accordingly, the second portion of the blood flow moving through the pulmonary branch 80 is provided into the pulmonary circulation 16 at a pressure different than the pressure of the blood provided from the systemic branch to the systemic circulation. In the illustrated embodiment, the graft 60 can be configured so that the systemic branch 70 and the pulmonary branch 80 have substantially the same flow resistance, so that blood flow is provided into the respective systemic and pulmonary systems at substantially the same pressures.

In the illustrated embodiment, the free end 82 of the pulmonary branch 80 is sutured or otherwise surgically attached to a portion of the patient's pulmonary artery 22 (FIG. 1) to provide a fluid and air tight interconnection therebetween. In other embodiments, the pulmonary branch 80 can be connected to another portion of the pulmonary circulation, such as another selected artery in the pulmonary circulation or other conduit attached to the pulmonary circulation.

The graft 60 can be made of one or more non-biological or biological materials compatible for use within a patient's body. Examples of non-biologic materials include Dacron or its equivalent, Gortex or its equivalent, woven polyester, or other non-biologic material suitable for use with a patient's body. Examples of biologic materials include human tissue, animal tissue (xenografts), biologically engineered tissues and conduits, or other biologic material suitable for use with a patient's body. In one embodiment, the entire graft 60 can be made of the same material. In another embodiment, the graft 60 can be made of more than one material. In some embodiments, the graft 60 can also be made of a material that can be combined with one or more selected chemicals or medications. As an example, the graft material can be coated or impregnated with a medication (such as heparin) to prevent graft thrombosis. In other examples, the graft material can be impregnated, coated or otherwise provided with an antibiotic, anticoagulant, and/or anti-inflammatory medications. Other embodiments can include a graft material that carries one or more other chemicals or medications. In yet other embodiments the graft 60 can be provided where the same or different medications are carried by the systemic and pulmonary branches 70 and 80.

In one embodiment, the graft 60 is constructed of a material (non-biological or biological) that allows the surgeon or other technician to cut or otherwise adjust the length or spatulation (i.e., the angular orientation of the end) of the inlet portion 62, the systemic branch 70, and/or the pulmonary branch 80 as needed. As an example, the graft 60 can be made of a commercially available material, such as Dacron or GorTex, which allows the surgeon or other technician to cut, shape, and/or otherwise modify the graft during surgery before the graft is secured in place, thereby ensuring that the graft has the proper length and spatulation for the patient. In other embodiments, the grafts 60 can be provided with predetermined shapes and sizes, so that the surgeon can select a suitable graft for the needs of the patient. The ends of the inlet portion 62, the systemic branch 70 and/or the pulmonary branch 80 can be structurally reinforced or provided with an increased thickness, for example, to provide a reinforced connection portion for sutures or other connection mechanisms. In other embodiments, the graft 60 can be configured to allow for the placement of an intracardiac shunt, such as a fenestrated patch, a stenting device or any other device that allows intracardiac shunting.

In the illustrated embodiment, the inlet portion 62, the systemic branch 70 and the pulmonary branch 80 of the graft form a generally Y-shape (FIG. 2) or T-shape (FIG. 3), although other shapes can be used. The graft 60 can be shaped and sized with any one of plurality size ratios of the inlet portion 62, the systemic branch 70 and the pulmonary branch 80 depending upon the characteristics of the blood-flow assist device 36, the differential flow resistance through the systemic and pulmonary branches, and/or the physiologic state of the patient. For example, it may be desirable to have a smaller pulmonary branch 80 of the graft than systemic branch 70, because the down stream pressures in those vascular beds are dramatically different. In other embodiments, the pulmonary branch may have a larger diameter or cross-sectional area than that of the systemic branch 70.

The graft 60 is configured to be paired with a single blood-flow assist device 36, such as an axial flow device, that can be adjusted to achieve the appropriate hemodynamics for the patient. As indicated above, the systemic and pulmonary branches 70 and 80 are shaped and sized with a relative size ratio that provides a resistance differential and the desired blood flow into each of the systemic and pulmonary circulations 16 and 18 for the patient (i.e., adult, child or infant) while using the single blood-flow assist device 36. The blood-flow assist device 36 and the graft 60 are surgically implanted (or otherwise attached to the patient), and the blood-flow assist device is configured so the blood flow rate can be adjusted to achieve the appropriate hemodynamics for the patient. In one embodiment, the blood-flow assist device 36 can be remotely adjusted (i.e., wirelessly) during or after surgery to adjust the flow rate as needed based upon the needs of the patient. For example, the blood-flow assist device 36 can be adjusted to provide blood flow into the graft 60 at a selected flow rate, which results in the blood flow through the systemic and pulmonary branches at the volumes and pressures needed for the patient. One of ordinary skill in the art will recognize that the flow rates can widely vary for different patients. For example, one patient may need a flow rate of approximately 100 ml/kg/min, and another patient may need to be 200 ml/kg/min. While the above flow rate numbers are provided as an example, the actual flow rates required by different patients could be much higher or lower depending on any given patients physiologic state. Nonetheless, the graft 60 is configured to work with the single blood-flow assist device 36 to simultaneously accommodate the range of flows needed for the systemic and pulmonary circulations. Accordingly, the graft can be different sizes to provide the selected resistance differential, and the resulting blood flow at different rates to the pulmonary and systemic circulations as necessary for the needs of the patient, thereby differentially distributing one source of blood to two different vascular beds.

For purposes of illustration, an example of a procedure for providing cardio-pulmonary assistance (including partial or full support) for a patient in accordance with one embodiment is described below. In this example, the patient with end stage heart failure (from any etiology) requires mechanical circulatory assist as either a bridge to recovery, a bridge to transplant or as destination therapy. Typically, the patient can not be managed medically and requires mechanical circulatory support in order to survive. Accordingly, the patient is then taken to the operating room for the procedure.

After the patient is placed under general anesthesia, arterial and venous lines are inserted percutaneously. The patient then undergoes routine prep and drape. A primary midline incision and median sternotomy are made. The thymus, if present, is removed. The pericardium is opened and suspended. Heparin is given. Routine aortic and direct bicaval cannulation is carried out. The patient is placed on cardiopulmonary bypass staying at approximately 37° C. The location for placement of the ends or outflows systemic and pulmonary branches 70 and 80 of the bifurcated graft 60 is assessed for placement on the respective aorta and the pulmonary artery. Once a site is chosen, the lie of the graft 60 is assessed. The end portion of the systemic branch 70 of the graft 60 is cut to length and spatulated. A side-biting clamp is placed on the aorta and a longitudinal incision is made with a scalpel into the aorta. Interrupted and pledgeted braided sutures are placed in a horizontal mattress fashion around the aortic incision and then passed through the cut end of the systemic branch 70. The graft is parachuted down and the sutures are tied. The side-biting clamp is removed and blood is allowed to back-fill the graft, which is then clamped.

A side-biting clamp is placed on the pulmonary artery and a longitudinal incision is made therein. The end portion of the pulmonary branch is then cut to length and spatulated. A running monofilament suture is used to attach (anastamose) the pulmonary branch of the graft to the pulmonary artery. The sidebiting clamp is removed and the graft is allowed to back-fill with blood and it is clamped. The apex of the heart is then lifted up and a coring device is used to make a circular incision in the left ventricular apex. Braided, pledgeted sutures are placed in a horizontal mattress fashion around the circular ventriculotomy. These sutures are placed through the sewing ring of the VAD inflow cannula, the cannula is parachuted down, and the sutures are then tied. The cannula is filled with blood and clamped. The graft 60 and the cannula are attached to the pumping mechanism and the pump is deaired. The pump is then turned on and the flow adjusted upward as the patient is weaned from cardiopulmonary bypass. In some embodiments, it may not be possible to leave the pump within the patient's chest. In those cases the pump can be placed in an alternate location (such as a preperitoneal pocket) or can be left in a paracorporeal location outside of the body. Once the appropriate hemodynamics are achieved, and the patient is weaned from bypass, the bypass cannulas would be removed from the heart. Hemostasis would then be obtained. Chest tubes are placed and the chest is closed. One of ordinary skill in the art will understand that this is only one example of a procedure in accordance with an embodiment, and that other procedures and/or other portions of the procedure, such as different surgical techniques can be used in other embodiments. For example, in some embodiments, there may be a need for intracardiac shunting of blood, such that an atrial level communication may be created with a synthetic patch with a hole in it. In other embodiments, it may be necessary to stop the heart by cross-clamping the aorta and giving a cardioplegia solution down the coronary arteries. When the heart is stopped an atrial level communication may be made and then controlled by sewing in a synthetic patch with a hole in it of a predetermined size. The cross-clamp would be removed from the aorta after the atrium was closed. This would all be done prior to weaning the patient from cardiopulmonary bypass.

The above-detailed embodiments of the invention are not intended to be exhaustive or to limit the invention to the precise form disclosed above. Specific embodiments of, and examples for, the invention are described above for illustrative purposes, but those skilled in the relevant art will recognize that various equivalent modifications are possible within the scope of the invention. For example, whereas steps are presented in a given order, alternative embodiments may perform steps in a different order. The various aspects of embodiments described herein can be combined and/or eliminated to provide further embodiments. Although advantages associated with certain embodiments of the invention have been described in the context of those embodiments, other embodiments may also exhibit such advantages. Additionally, not all embodiments need necessarily exhibit such advantages to fall within the scope of the invention.

Unless the context clearly requires otherwise, throughout the description and the claims, the words “comprise,” “comprising,” and the like are to be construed in an inclusive sense as opposed to an exclusive or exhaustive sense, i.e., in a sense of “including, but not limited to.” Additionally, the words “herein,” “above,” “below,” and words of similar import, when used in this application, shall refer to this application as a whole and not to any particular portions of this application. Use of the word “or” in reference to a list of items is intended to cover a) any of the items in the list, b) all of the items in the list, and c) any combination of the items in the list.

In general, the terms used in the following claims should not be construed to limit the invention to the specific embodiments disclosed in the specification unless the above-detailed description explicitly defines such terms. In addition, the inventor contemplates various aspects of the invention in any number of claim forms. Accordingly, the inventor reserves the right to add claims after filing the application to pursue such additional claim forms for other aspects of the invention.

1. A bifurcated blood flow assist system for use with a circulatory system of a patient, the circulatory system including a heart, a pulmonary circulation, and a systemic circulation, the system comprising:
a blood-flow assist device coupled to the heart and having a blood flow inlet that receives a blood flow from the heart, and having a blood flow outlet; and
a bifurcated graft that connects to the pulmonary circulation and the systemic circulation, the bifurcated graft comprising:
an inlet portion connected to the blood flow outlet of the blood-flow assist device and configured to receive the a blood flow therethrough from the blood-flow assist device;
a systemic portion connected to the inlet portion and configured to receive a first portion of the blood flow from the inlet portion, the systemic portion having a systemic outlet that connects to a first artery in the patient's systemic circulation to direct the first portion of the blood flow directly into the first artery at a first blood flow rate and at a first blood pressure suitable with the systemic circulation; and
a pulmonary portion connected to the inlet portion and that receives a second portion of the blood flow from the inlet portion, the pulmonary portion having a pulmonary outlet that connects to a second artery in the patient's pulmonary circulation to direct the second portion of the blood flow directly into the second artery substantially simultaneous with delivery of the first portion of the blood flow into the first artery and at a second blood flow rate different than the first blood flow rate and at a second blood pressure different than the first blood pressure and suitable with the pulmonary circulation.

2. (canceled)

3. The graft of claim 1 wherein the inlet portion, the systemic portion and the pulmonary portions are arranged in a Y-shape or a T-shape.

4. The graft of claim 1 wherein at least one of the systemic portion and the pulmonary portion are configured so the length and spatulation can be modified.

5. The graft of claim 1 wherein the graft is made from a non-biological material.

6. The graft of claim 1 wherein the graft is made of at least one of Dacron, Gortex, polyester, or equivalents.

7. The graft of claim 1 wherein the graft is a xenograft or a biologically engineered material.

8. The graft of claim 1 wherein the inlet portion of the graft is mechanically locked to a portion of the blood-flow assist device.

9. The graft of claim 1 wherein the inlet portion is integrally connected to the blood-flow assist device.

10. The graft of claim 1 wherein the inlet portion has a first lumen with a first cross-sectional area, the systemic portion has a second lumen with a second cross-sectional area substantially equal to or less than the first cross-sectional, and the pulmonary portion has a third cross-sectional area less than the second cross-sectional area.

11-17. (canceled)

18. A ventricular assistance system for use with a heart, a systemic circulation, and a pulmonary circulation of a patient, comprising:
a blood-flow assist device having a first inlet portion and an outlet portion, the first inlet portion being coupleable to the heart and configured to receive a flow of blood therefrom, and a tubular graft shaped and sized to be surgically implanted in the patient, the graft comprising:
a second inlet portion coupled to the outlet of the blood-flow assist device and configured to receive the blood flow therethrough;
a systemic branch coupled to the second inlet portion and configured to be connected to the systemic circulation of the patient to direct a first portion of the blood flow into the systemic circulation at a first blood flow rate and at a first blood pressure suitable with the systemic system; and
a pulmonary branch coupled to the inlet portion and configured to be connected to the pulmonary circulation of the patient to direct a second portion of the blood flow into the pulmonary circulation substantially simultaneous with delivery of the first portion of the blood flow into the systemic circulation and at a second blood flow rate different than the first blood flow rate and at a second blood pressure suitable with the pulmonary circulation,.

19. The system of claim 18 wherein the blood-flow assist device is a ventricular assist device.

20. The system of claim 18 wherein the blood-flow assist device includes an axial pump.

21. The system of claim 18 wherein the systemic and pulmonary branches are shaped and sized so the systemic branch has a first flow resistance, and the pulmonary branch has a second flow resistance different than the first flow resistance.

22. The graft of claim 18 wherein the inlet portion has a first lumen with a first cross-sectional area, the systemic branch has a second lumen with a second cross-sectional area substantially equal to or less than the first cross-sectional area, and the pulmonary branch has a third cross-sectional area less than the second cross-sectional area.

23. The system of claim 18 wherein the graft is fully implantable in the patient.

24. The system of claim 18 wherein the graft is made from a non-biological material.

25. The system of claim 18 wherein the second inlet portion is integrally connected to the blood-flow assist device.

26-35. (canceled)

36. The system of claim 18 wherein the systemic branch is surgically connected directly to a first artery in the systemic circulation, and the pulmonary branch is surgically connected directly to a second artery in the pulmonary circulation.

37. The system of claim 18 wherein the second inlet portion is integrally connected to the systemic branch and the pulmonary branch.

38. The system of claim 18 wherein the second inlet portion is mechanically locked to the blood-flow assist device.

39. The system of claim 18 wherein the second inlet portion is integrally connected to the blood-flow assist device.
